(19) United States
(12) Patent Application Publication
(10) Pub. No.: US 2022/0133783 A1
(43) Pub. Date: May 5, 2022

(54) ANTIVIRAL COMPOSITION AND USE OF THE SAME

(71) Applicant: SALVACION USA INC., Englewood Cliffs, NJ (US)

(72) Inventors: Abdul GAFFAR, Lakewood Ranch, FL (US); Yeong Wan CHO, Palisades Park, NJ (US); Sei Young YUN, Daejeon (KR)

(73) Assignee: SALVACION USA INC., Englewood Cliffs, NJ (US)

(21) Appl. No.: 17/576,098

(22) Filed: Jan. 14, 2022

Related U.S. Application Data
(63) Continuation-in-part of application No. PCT/US20/55772, filed on Oct. 15, 2020.
(60) Provisional application No. 63/103,881, filed on Aug. 31, 2020.

(51) Int. Cl. A61K 33/34; A61K 9/107; A61P 31/12; A61K 47/16

(52) U.S. Cl. CPC A61K 33/34; A61K 47/16; A61P 31/12; A61K 9/1075

(57) ABSTRACT

Provided are an antiviral composition containing a cationic antiviral agent (cationic agent) and a copper salt to prevent, control or treat viral infections in a mammal, particularly in the nasopharyngeal and throat areas of humans and animals, and a method of preventing, controlling or treating viral infections in a mammal using the same.

### CROSS-REFERENCE TO RELATED APPLICATIONS

This application is a Continuation-in-Part of PCT Application No. PCT/US2020/055772, filed on Oct. 15, 2020, which claims priority to and the benefit of U.S. Provisional Patent Application No. 63/103,881, filed on Aug. 31, 2020, the disclosure of which is incorporated herein by reference in its entirety.

### FIELD OF THE INVENTION

This invention relates to an antiviral composition containing a cationic antiviral agent (cationic surfactant) and a copper salt to control viral infections in the nasopharyngeal and throat areas of humans and animals.

### BACKGROUND OF THE INVENTION

Acute Respiratory Syndrome (SARS) virus and Covid virus were first found in China and rapidly spread over Asia, Europe, North America, etc. Current evidence suggests that the virus spreads between people through direct, indirect (through contaminated objects or surfaces), or close contact with infected people via mouth and nose secretions. These include saliva, respiratory secretions or secretion droplets.

Worldwide outbreaks of Covid virus infection illustrate the complexity of effective treatments. Recently, worldwide outbreaks of Covid and SAR infections led to an urgent search for optimum tools to limit the spread of diseases.

Coronavirus family includes alpha coronaviruses 229E, NL63, Beta 0043, HKU1, and human corona viruses are MER 6-COV C Middle East respiratory Syndrome. SAR-COV (beta coronavirus that cause respiratory syndrome SARS and SAR-COV-2 and the novel coronavirus that cause Coronavirus 2019, Covid-19.

People around the world commonly get infected with human coronavirus 229E, NL63, 0043, and HKU1. It is believed in the art that Covid-19 is a good experimental model for determining the biological activity of a synergistic combination against Covid viruses.

Cationic surfactants' anti-bacterial functions are well known in the art for a variety of applications as anti-germ agents, such as water/oil emulsion in nanoparticles as disclosed in U.S. Pat. No. 8,877,208.

A copper salt has been used in fighting infections (see Gadi, Borkov. Current Chemical Biology 2012, 6; Borkov, G et al 2007, Antimicrobial Agents Chemotherapy Vol 51 page 2605.

Cationic surfactants derived from lauric acid and arginine, in particular, the ester of lauramide of arginine monohydrochloride, also known as ethyl-N-alpha-lauroyl-L-arginate HCl, lauramide arginine ethyl ester, lauric arginate ethyl ester, or ethyl lauroyl arginine hydrochloride (ELAH), may be used for protection against the virus. The ELAH and its derivatives are described in WO 2008/014824 and the disclosure is incorporated herein by reference in its entirety.

Among the most common cationic antibacterial or antiviral is a quaternary ammonium compound disclosed in U.S. Pat. Nos. 2,984,639; 3,325,402; 3,431,208 and British Patent No. 1,319,396, each of which is incorporated herein by reference in its entirety.

Safe and effective antiviral products for the treatment of viral infections are urgently needed, particularly considering the current worldwide break of Covid-19. There is an urgent need in the art for safe and effective new treatments for viral infections.

### SUMMARY OF THE INVENTION

The present inventors have found that a composition comprising a cationic antiviral agent, a copper salt and water shows a surprising, remarkably strong synergistic antiviral activity. Particularly, a composition comprising a cationic surfactant, ELAH or benzalkonium chloride (BAC), in combination with a copper salt showed a synergistically improved antiviral activity, which is unexpected from each of the components when they used alone. Further, the present inventors have found that the composition, when it is applied to the nasal cavity, forms a physical barrier on the surface of the cavity, particularly the surface of the nasopharynx, and protects the virus from adhering to mucosal tissue of the nasal passages, thus stopping further transmission for infection. Accordingly, one object of the present invention is to provide an antiviral composition, particularly an antiviral microemulsion composition comprising an effective amount of a cationic antiviral agent, particularly an arginine ester cationic surfactant, a copper salt and a solvent. Another object of the present invention is to provide a method of preventing, inhibiting or treating a viral infection in a subject in need thereof comprising applying the composition to the subject, particularly to the nasal cavity of the subject.

One aspect of the present invention relates to an antiviral composition comprising a cationic antiviral agent, a copper salt and water.

The cationic agent in the composition may be in an amount of 2 ppm to 20,000 ppm, and the copper salt may be in an amount of 1 ppm to 10,000 ppm. The solvent in the composition may be selected from one or more of water, alcohol, propylene glycol, ethyl acetate, methyl isobutyl ketone, acetone, tetrahydrofuran, isopropyl ether, and a combination thereof. The cationic agent is selected from the group consisting of ethyllauroyl arginate, a quaternary ammonium compound, benzalkonium chloride, benzethonium chloride, methylbenzethonium chloride, cetalkonium chloride, cetylpyridinium chloride, cetrimonium, guanidine, and a combination thereof. The copper salt comprises a gluconate, a citrate, an acetate, an amino acid or a peptide.

The antiviral composition may comprise 0.01% to 20% of a plasticizer selected from glycol, glycerin, xylitol, ethanol, and a combination thereof.

In the antiviral composition, the cationic agent and the copper meets Equation 1 described below. In the equation, FICI means Fractional inhibitory Concentration (FIC) Index, FICA means the FIC of agent A, FICB means the FIC of agent B. Herein, agent A is the cationic agent and agent B is the copper agent.

FICI=FICA+FICB. [Equation 1]:

In the equation, FICA=[CA]sy/[CA]al and FICB=[CS]sy/[CS]al. [CA]al is a minimum inhibitory concentration (MIC) of the cationic agent, and [CS]al is a minimum inhibitory concentration (MIC) of the copper salt, [CA]sy is a minimum inhibitory concentration (MIC) of the cationic agent where the cationic and the copper agents are used at the same time, and [CS]sy is a minimum inhibitory concentration (MIC) of the copper salt where the cationic the a copper salt are used at the same time.

In the antiviral composition, the fractional inhibitory index (FICI) is less than 0.5. FICI<0.5 indicates synergistic, FICI of >1 indicates additive, and FICI of >2 indicates indifferent (Hollander et al: Antimicrobial agents Chemotherapy 1998, Vol. 42, pages 744-748).

The antiviral composition has a pH between pH 4 and pH 8. The pH may be between 4 and 6.5, between 4 and 5, between 4.4 and 5, or between 4.6 and 5.

The antiviral composition may be formulated into or be in the form of, for example, a nasal spray, a nasal gel, an aerosol, a throat lozenge, a gargle, an oral strip, a topical formulation, or an external use formulation. However, it is not limited to the formulations.

Where the antiviral composition is applied to a surface, it may be used in an amount of 0.01 to 100 mg/dm², preferably 0.5 to 50 mg/dm², and more preferably 1 to 19 mg/dm².

Another aspect of the invention relates to a method for preventing, inhibiting, controlling or treating bacterial or viral infections in a subject in need thereof, comprising administering or applying the composition containing a cationic antiviral and a copper salt, as described above, to a subject in need thereof, particularly to the nasal cavity of the subject, more particularly to the nasopharyngeal or throat surface of humans and animals.

In one embodiment, the composition is pre-administered to the subject for prophylactic effect, wherein the composition reduces viral entry and the cytopathic effect thereof. The composition, when it is administered nasally, is retained in the nasal cavity for a prolonged time, for example, 2 hours or longer, and reduces viral entry and the cytopathic effect of the virus.

Both the foregoing summary of the invention and the following brief description of the drawings and the detailed description of the invention are exemplary and explanatory and are intended to provide further details of the invention as claimed. Other objects, advantages, and novel features will be readily apparent to those skilled in the art from the following detailed description of the invention. The present invention will be described in detail as follows.

### BRIEF DESCRIPTION OF THE DRAWINGS

FIG. 1 illustrates antiviral effect of Solution1 against rVSG-dG 2019-CoV-2-18AA S in Vero cells on day 1.

FIG. 2 illustrates antiviral effect of Solution2 against rVSG-dG 2019-CoV-2-18AA S in Vero cells on day 1.

FIG. 3 illustrates SEP imaging photos of MRC-5 cells alone as control after 48-hour incubation at 35° C. A: 10 μm; B: 3 μm.

FIG. 4 illustrates SEP imaging photos of MRC-5 cells after 2-hour exposure to human coronavirus 229E (10⁻³ dilution) (A) and after rinsing to remove virus and 48-hour incubation (B). A: 20 μm; B: 5 μm.

FIG. 5 illustrates SEP imaging photos of MRC5 cells after 10-minute exposure to 10 μg/mL (10⁻² dilution) of active ELAN (non-cytotoxic concentration) (A), and after 2-hour exposure to coronavirus 229E (10⁻³ dilution) and 48-hour incubation (B). A: 20 μm; B: 5 μm.

### DETAILED DESCRIPTION OF THE INVENTION

The antiviral composition provided herein comprises a cationic agent with antiviral activity, a copper salt and a solvent. The cationic agent may be selected from ethyl lauroyl arginate, quaternary ammonium compounds, and guanidine compounds.

The cationic antiviral agent may be a cationic surfactant, which is derived from lauric acid and arginine, in particular, the ester of lauramide of arginine monohydrate, hereafter named, ELAN, and may be used for protection against virus. Details of ELAN and its derivatives are described in WO 2008/014824, the content of which is incorporated herein by reference in its entirety.

The cationic antiviral agent may be quaternary ammonium compounds, which are disclosed in U.S. Pat. Nos. 2,984,639; 3,325,402; 3,431,208 and British Patent No. 1,319,396, each of which being incorporated herein. The quaternary ammonium compounds of the cationic antiviral may include those in which one or two substitutions of the quaternary nitrogen has a carbon chain length of typically alkyl groups 8 to 20, typically 10 to 18 while the remaining substituents have lower carbon atoms typically alkyl or benzyl groups such as 1 to 7 atoms, typically methyl or ethyl groups. These include benzalkonium chloride, cetyl pyridinium chloride.

The cationic antiviral may be guanidine compounds which are disclosed in German Patent application No. P 2,233,383 and it is incorporated herein.

The copper salt used is a copper salt releasing copper ions in water. The copper salt comprises a gluconate, a citrate, an acetate, amino acids, peptides and complexes of copper/polymer.

Non-limiting examples for copper (II) salts include Copper (II) sulfate, Copper (II) chloride, Copper (II) hydroxide, Copper (II) perchlorate, Copper (II) selenite, Copper (II) sulfide), Copper (II) thiocyanate, Copper (II) triflate, Copper (II) tetrafluoroborate, Copper (II) acetate triarsenite (Paris Green), Copper (II) benzoate, C (Scheele's Green), Copper (II) chromite, Copper (II) gluconate, Copper(II) peroxide, Copper (II) usnate.

A copper salt of the amino acids and peptides are disclosed by P. A. Kober and K. Surguira (J. Bio. chem., vol X111 no 1 pages 1-11), the content of which is incorporated herein, and it may include the salts of glycine, alanine, aminobutyric acids, valine, leucine, isoleucine and di- and polypeptides of amino acids.

Copper polymeric complexes such as acrylic acids, polymers, oligomers, copolymer of maleic acids and/or anhydrides and of olefin having one or more atoms of carbon atoms per molecule may be used. The preferred are polymeric polymaleate, polymethyl methacrylate, vinylmethy ether copolymer and other carboxylic polymer disclosed in U.S. Pat. No. 4,217,343, the content of which is incorporated herein by reference.

The most preferred combination of the components is ELAH or Benzalkonium chloride with a copper salt to achieve synergistic antiviral effects.

The composition further comprises 0.01%-20% of a plasticizer, wherein the plasticizer may be selected from glycol, glycerin, ethanol, and a combination thereof.

It is preferred to dissolve the compounds directly before use in one of the preferred solvents of food-grade water, ethanol, glycerin, propylene glycol and a mixture of glycol with water. If the treatment shall be performed at specific pH values (pH 4 to pH 8), the use of a corresponding buffer solution may be recommended. On the other hand, the synergistic combination can be easily used as a semisolid or a solid. Surfaces shall be protected, for instance, the surface of masks, solid surfaces on the furniture, protective clothes, etc.

The present invention relates to the use of the combination of a cationic surfactant, ELAH or BAC, and a copper salt to achieve a synergistic antiviral effect against virus infections.

The present invention furthermore relates to the application of the cationic surfactants of formula with a copper salt to a subject in need thereof, particularly animals or human beings directly, for prophylactic, inhibiting or therapeutic treatment of virus diseases. A “subject in need” refers to a human or animal at risk of a viral infection, or which has contracted a viral infection.

The cationic surfactants of the formula disclosed in WO 2008/0014824 plus a copper salt may be applied to a surface as a solution. This is the easy and suitable manner of treating the surface of the ground, cars, animals and people. For other applications, it may be more suitable to apply the cationic surfactants plus a copper salt as a solid which may be equally effective.

The treatment of product to avoid any kind of virus infection might involve the presence of a concentration of the cationic surfactants of the formula, ELAH or BAC with a copper salt, more in particular according to the embodiment of ELAH or BAC of around 2 to 20,000 ppm plus a copper salt 1 to 10,000 ppm product to be protected, preferably a concentration of 100 to 10,000 ppm and more preferably 200 to 2000 ppm. This is a similar concentration as has been described for achieving the microbiocidal action. Products to be treated with the above-indicated range of concentrations of the cationic surfactants plus a copper salt are for instance food products or cosmetics.

The treatment of surfaces that are infected with viruses, such as the surface of food preparations, the surface of cosmetics, ground surface, the surface of any kind of vehicles, and the surface of any equipment used in the handling of animals infected with the virus, requires the presence of cationic surfactant ELAH or BAC plus a copper salt, in particular according to a preferred embodiment of ELAH or BAC plus a copper salt of level which is sufficient to achieve the wanted antiviral activity at such surfaces. Such level of concentration would be expected 2 to 20,000 ppm, more preferred 100 to 10,000 ppm and even more preferred 100 to 10000 ppm and even more preferred 200 to 2000 ppm, containing the surfactant plus a copper salt of claims, according to the preferred containing ELAN, BAC and a copper salt. These concentrations are given in terms of the concentration of a solution containing the cationic surfactant plus a copper salt which is applied to the surfaces to be treated. If surfaces are treated with solid preparation of the cationic surfactant of the formula, the amount which is applied shall be such that the amount of the cationic surfactant of ELAN or BAC plus a copper salt shall be in the range of 0.01 to 100 mg/dm², preferably an amount of 0.5 to 50 mg/dm², and more preferably an amount of 1 to 19 mg/dm².

The treatment of liquid preparations such as drinking fluids or natural sources of water such as lakes or ponds requires the presence of the cationic surfactants, more in particular, according to the preferred embodiment of ELAN or BAC plus a copper salt at a concentration of a level which is sufficient to achieve the wanted antiviral in the drinking fluid or water. Such level of concentration would be expected in the range of 0.2 to 20,000 ppm, more preferred 2 to 15,000 ppm, even more preferred 100 to 10,000 ppm and most preferred 200 to 2,000 ppm containing the cationic surfactants ELAN or BAC with copper salt according to the preferred embodiment containing ELAN or BAC plus copper salt. These concentrations are provided in terms of the concentration of the cationic surfactant in the liquid or the water to be treated.

The treatment of animals or humans implies the application of the cationic surfactant in a manner which is suitable for the application of the compounds used according to one aspect of the invention. The compounds may be applied topically, such as rectal application, external application to the skin or trans-nasal application. The formulations to be applied may be a conventional formulation, such as capsules, microcapsules, tablets, granules, powder, pills, ointments, suppositories, oral strips, suspensions, syrups, emulsions, liquids, sprays, inhalants, and nose drops. Preferably, it is a spray, solution, or microemulsion.

In one embodiment, the antiviral composition is microemulsion. A microemulsion is a thermodynamically stable fluid, the particle size of which may range from about 10 nm to 300 nm. Because of the small particle sizes, microemulsions appear as clear or translucent solutions.

The microemulsion composition according to the present invention may have particle sizes of 10 to 300 nm, preferably 10 to 200 nm, 10 to 180 nm, 10 to 60 nm, 20 to 40 nm, or 25 to 40 nm.

The microemulsion composition was characterized for size and size distribution using several techniques, dynamic light scattering (DLS), asymmetric-flow field flow fractionation (AF4), and light scattering (DynaPro). In addition, particle concentration by light scattering, zeta potential, ELAH concentration by reversed phase high performance liquid chromatography (RP-HPLC), and total and free copper as well as the presence of metal impurities by inductively coupled plasma mass spectrometry (ICP-MS) were also measured.

In one embodiment, the hydrodynamic size measured by Dynamic light scattering (DLS) in 10 mM NaCl (zeta potential conditions) and PBS (to mimic physiological ionic strength) showed several size populations and that the majority of particles have about 14 nm particles. The particle size measured with the light scattering (DynaPro®) in another embodiment showed that the majority of particles of the microemulsion composition have an average particle size of 14.8±2.3 nm, with an average particle concentration of 1.46±0.97 E+13 particles/ml.

The size distribution assessed using asymmetric-flow field flow fractionation (AF4) coupled with MALS and DLS detectors showed two size populations, the first peak having the hydrodynamic size ranging from 20 to 40 nm (25.4 nm on average) and the second peak having the hydrodynamic size ranging from 60-170 nm (93.6 nm on average). Upon incubation with plasma, the first peak had the hydrodynamic size ranged from 25-40 nm and the second peak had the hydrodynamic size ranged from 35-180 nm (81.1 nm in average).

The microemulsion composition was evaluated for potential contamination with endotoxin and beta-glucans. Endotoxin was assayed using the kinetic turbidity Limulus Amebocyte Lysate (LAL) assay, and beta-glucans were assayed using the commercial Glucatell assay. Both endotoxin and beta-glucan levels were below the assay detection limits, and therefore, are not expected to pose a safety concern.

The above-mentioned formulations may be prepared according to conventional methods using various organic or inorganic carriers, excipients or additives conventionally used for topical or external formulations, such as plasticizers, pH adjusters, thickeners, fragrances, emulsifiers, preservatives, stabilizers (such as citric acid, sodium citrate, acetic acid), suspending agents (such as methylcellulose, polyvinylpyrrolidone, aluminum stearate), dispersing agents (such as hydroxypropylmethyl cellulose), diluents (such as water), base waxes (such as cacao butter, white petrolatum, polyethylene glycol) or other suitable ones.

Non-limiting examples of the plasticizers include glycol, glycerin, xylitol, ethanol, or a combination thereof. The plasticizers may be used in an amount of 0.01% to 20 wt. %, preferably 0.5 to 10 wt. %, and more preferably 5 wt. %.

Non-limiting examples of the preservatives include phenoxyethanol. The preservatives may be used in an amount of 0.05 to 2.5 wt. %, preferably 0.05 wt. %.

Non-limiting examples of the humectants include 1,2 hexanediol. The humectants may be used in an amount of 0.1 to 10 wt. %, preferably 5 wt. %.

Non-limiting examples of the pH adjusters include sodium hydroxide or citric acid. The pH adjusters may be used in an amount to adjust the pH of the composition to be in the range of pH 4.5 to 6.5.

Non-limiting examples of the thickeners include PVP (K 90). The thickeners may be used in an amount of 1 to 10 wt. %, preferably 1 to 3 wt. %, and more preferably 1 wt. %.

Non-limiting examples of the fragrances include lavender. The fragrances may be used in an amount of 0.1 to 1 wt. %, preferably 0.01 wt. %.

Non-limiting examples of the emulsifiers include PEG-40 hydrogenated castor oil. The emulsifiers may be used in an amount of 0.1 to 1 wt. %, preferably 0.01 wt. %.

The composition of the present invention may be applied 1 to 4 times per day, or as needed.

### EXAMPLES

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1

VSV-Pseudo Type Neutralization Assay for SARS-CoV2

IBT (Integrated Biologic Testing) conducted the study using established a VSV Neutralization assay similar to the system IBT and others have previously reported for filoviruses and SARS-CoV2. Briefly, VSV lacking G has been pseudo typed with SARS-CoV2 Spike protein and produced in HEK293T cells. This system contains luciferase reporter gene which is used for assay readout.

Specifically, four dilutions of the test combination of ELAH or BAC with copper gluconate, 200, 100, 50 and 25 μg/ml and controls were prepared and mixed with VSV virus in a ratio of 1:1 for 1 hour at room temperature followed by incubation over Vero cells at 37° C. The cells were lysed the following day and luciferase activity was measured to assess antiviral effect of the test compound to block viral entry in the Vero cells. All samples were run in triplicate. Data analysis was conducted using XLFit and Graphed pad Prism.

Synergistic Effect Between the Cationic Agent (e.g., ELAH or BAC) and Copper Estimated by the Fractional Inhibitory Index (FICI)

It was found that the antiviral effect (against Covid 19) of the combination is higher, when a copper salt and a cationic antimicrobial agent are used in combination, than the summation of separate use of a copper salt and a cationic antimicrobial agent. The antiviral composition containing the cationic agent and the copper salt in combination, or when the cationic agent and the copper salt are used simultaneously, meets Equation 1:

[Equation 1] FICI=FICA+FICB, wherein FICA=[CA]sy/[CA]al, FICB=[CS]sy/[CS]al, wherein [CA]al is the minimum inhibitory concentration (MIC) of the cationic agent (ex, ELAH or BAC) alone respectively, CS]al is the minimum inhibitory concentration (MIC) of the copper salt alone respectively, [CA]sy is the minimum inhibitory concentration (MIC) of the cationic agent (ex, ELAH or BAC) when the cationic and the copper agents are used at the same time, [CS]sy is the minimum inhibitory concentration (MIC) of the copper agents when the cationic the copper agents are used at the same time.

The fractional inhibitory index (FICI) of the composition is less than 0.5, which indicates that the composition has a synergistic antiviral effect. Per established principles of synergism between two active agents, if the fractional inhibitory concentration of two agents, when added, is less than 0.5, synergism is demonstrated. That is, FICI<0.5 is synergistic, FICI of >1 is additive, and FICI of >2 is indifferent (Hollander et al: Antimicrobial agents Chemotherapy 1998, Vol 42 page 744-748).

### Example 2

Spray Solutions and Evaluation of Antiviral Effects Thereof

To evaluate the antiviral effect of the composition of the present invention, Solution 1 was prepared with the active ELAH and other components as shown below in the table, according to a method known in the art for preparing a spray solution.

Solution1:

| Ingredient | Function | Dosage | |
|---|---|---|---|
| ELAH (20%) | Active | 0.1-2 | w/v % |
| Copper Gluconate | | 0.001-1.0% | |
| Glycerin | plasticizer | 1-30 | g |
| Xylitol | plasticizer | 1-15 g (5.00 w/v %) | |
| Phenoxyethanol | Preservative | 0.01-0.5 | g |
| 1,2 hexanediol | Humectant | 0.2-5 | g |
| Sodium Hydroxide | pH adjuster | q.s. | |
| Citric acid | pH adjuster | q.s. | |
| PVP (K 90) | Thickener | 0.1-5 | g |
| Lavendar | Fragrance | q.s. | |
| PEG-40 Hydrogenated Castor Oil | Emulsifier | 0.01-3 | g |
| Purified Water | Solvent | to 100 | ml |

| | | |
|---|---|---|
| Total | 100 | ml |

| | |
|---|---|
| pH | 5.0 ± 1.5 |
| Appearance | Transparent Liquid |

Solution2:

| Ingredient | Function | Dosage | |
|---|---|---|---|
| Benzalkonium Chloride (50%) | Active | 0.01-5.0 g (w/v %) | |
| Copper Gluconate | | 0.001-1.0% | |
| Glycerin | plasticizer | 1-30 | g |
| Xylitol | plasticizer | 1-15 g (5.00 w/v %) | |
| Phenoxyethanol | Preservative | 0.01-0.5 | g |
| 1,2 hexanediol | Humectant | 0.2-5 | g |
| Sodium Hydroxide | pH adjuster | q.s. | |
| Citric acid | pH adjuster | q.s. | |
| PVP (K 90) | Thickener | 0.1-5 | g |
| Lavender | Fragrance | q.s. | |
| PEG-40 Hydrogenated Castor Oil | Emulsifier | 0.01-3 | g |
| Purified Water | Solvent | to 100 | ml |

| | | |
|---|---|---|
| Total | 100 | ml |

| | |
|---|---|
| pH | 5.0 ± 1.5 |
| Appearance | Transparent Liquid |

Antiviral Activity Test Results:

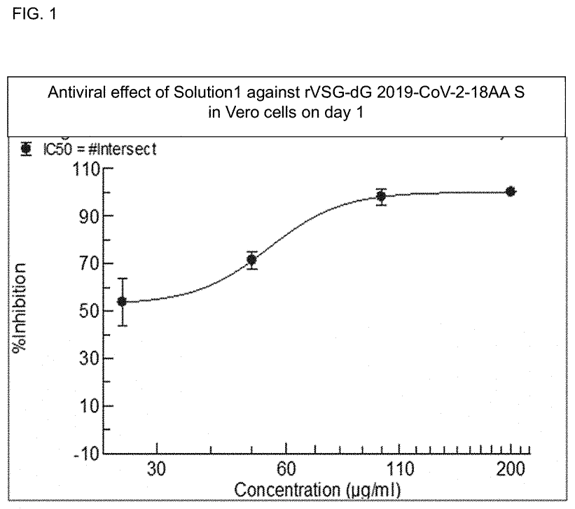

The results of viral inhibition of SV Covid-19 by ELAH/copper gluconate combination (Solution 1) are illustrated in FIG. 1, at the concentration of 25, 50, 100 and 200 μg/ml. The reported effect of ELAH by itself on virus inhibition is 300 μg/ml (WO 2008/014824) and copper by itself is 300 μg/ml (Sagripanti, J C et al Applied environ. microbiol: 1993: vo159:4374-4376). Therefore, FICI for ELAH/copper combinations: ELAH 30/Cu30/300=0.10+0.10=0.20 (inhibition conc. is at 30 ppm) is less than 0.5, which indicates synergism between ELAH and copper against SAR-Covid.

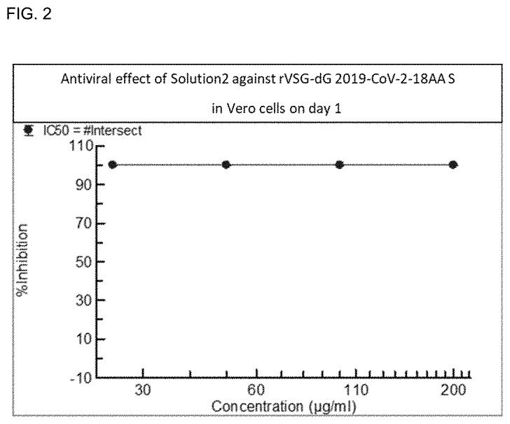

The results of viral inhibition of SV Covid-19 by BAC plus a copper salt (Solution 2) are illustrated in FIG. 2. The figure shows BAC plus copper gluconate had 100 percent inhibition on SV CoVid2 at 20 μg/ml. However, BAC by itself has been reported to have antiviral effect at 100 μg/ml (Eric G Romanoswki et al. J. occul. Phamacol therapy. 2019: vo135: pages 311-314). Therefore, FICI for BAC/copper combination: BAC 20/100+Copper 20/300=0.2+0.07=0.27 is lower than 0.5, which indicates synergism between BAC and the copper salt against SAR-CoVid 2.

### Example 3

Characterization of the Nasal Spray Inhibition of Coronavirus 229E Binding to MRC-5 Cells (Solution 1 in Example 2: COVIXYL-V™).

The efficacy of active ELAH on altering cell susceptibility to viral entry was assessed using SEM, under the condition:
i. Viral strain and amplification number: Human coronavirus 229E (ATCCO VR-740™)-amplification number: 1
ii. Cell line: MRC-5 (ATCC® CCL-171™)—passage number: 9
iii. Viral cell culture medium: Eagle's Minimum Essential Medium (EMEM) 2%, Fetal Bovine Serum, 1% penicillin/streptomycin.
iv. Product test concentrations: Based on findings from Phase 2a, COVIXYL-V™ 0.1% (1000 μg/mL) was diluted in viral cell culture media to Dilutions of 10⁻² (10 μg/mL) and 10⁻³ (1 μg/mL active concentration).
v. Diluent used for test item: Viral cell culture media
vi. Contact time(s): 10 min for test product
vii. Incubation conditions: 37° C.±2° C. and 5% CO₂ (MRC-5 cell culture), 35° C.±2° C. and 5% CO₂ (Viral barrier studies)
viii. Methods of assessment: High Resolution Scanning Electron Microscopy, University of Wyoming.

Viral Barrier Studies:

MRC-5 (ATCC® CCL-171™) cells (passage number: 9) were seeded approximately 1×10⁵ cells/mL to CELLTREAT® 4 chamber cell culture slides (229164) and incubated 37° C.±2° C. and 5% CO₂ for 4 days until 80% to 90% confluence. At time zero of experimentation, active ELAH dilutions 10⁻² and 10⁻³ 750 μL (total volume) were applied to cells in the chambers and incubated 37° C.±2° C. and 5% CO₂ for 10 minutes, then culture media containing unbound test product was removed and prepared human coronavirus 229E dilutions (10⁻², 10⁻³ and 10⁻⁴) in viral cell culture media were applied to wells followed by a 2-hour incubation at 35° C.±2° C. and 5% CO₂. Following incubation, viral cell media was aspirated to remove unbound virus, cells rinsed and incubated 35° C.±2° C. and 5% CO₂ for 48 hours with viral culture media. After 48-hour incubation, chamber cell culture slides were imaged via bright field microscopy (results not provided herein) and processed for SEM fixation.

SEM Imaging:

After samples underwent fixation, they were placed in a Kinney Vacuum KSE-2A-M Evaporator under 10⁴ Torr vacuum for 24 hours, then sputtered with a 5 nm thick gold coat using a Model 30000 Ladd Research Industries apparatus. Secondary electron and backscattered electron images were collected on a Quanta 250 Scanning Electron Microscope under 10⁻⁵ Torr vacuum using an accelerating voltage of 5 kV and spot sizes of 2 and 3. Electronic alignments on the electron gun (Gun Alignment, Final Lens Aperture Alignment, and Stigmator Alignment) were performed prior to imaging to optimize resolution.

Results:

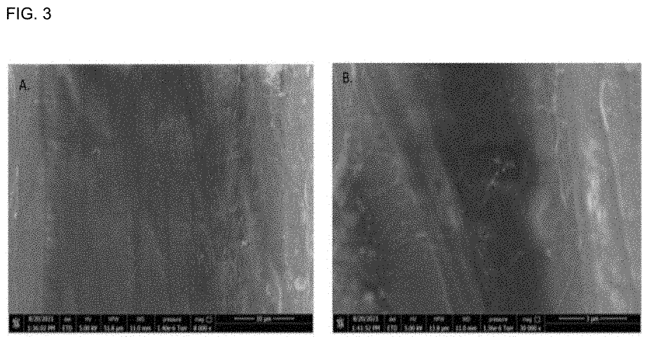

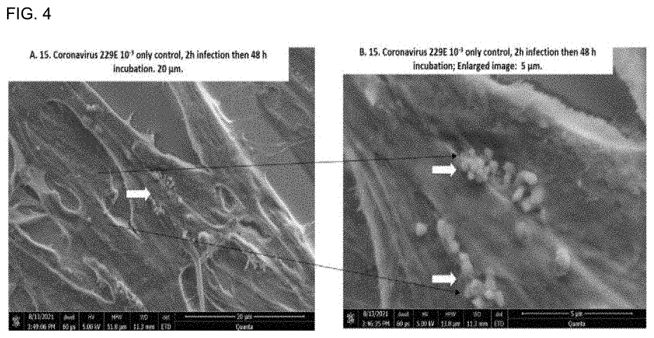

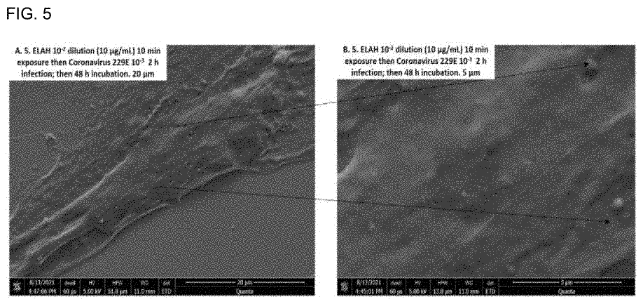

As shown by FIGS. 3 to 5, the SEM microscopy studies indicated that pre-treatment with active ELAN 10 μg/mL inhibited human coronavirus 229E binding and replication in the MRC-5 cell line. These data indicate that 10 minutes of pre-treatment of MRC-5 cells with active ELAN 10 μg/mL prior to human coronavirus 229E, reduces viral entry and the cytopathic effects caused by the virus after 48 hours of incubation compared to controls. The composition according to the present invention made the active ELAN to retain in nasal passage for 2 hours and longer. Accordingly, the composition of the present invention is effective for prophylactic effect, preventing the infection by the virus.

It was found that the nasal spray creates a physical barrier on the nasal surface, preventing the virus from adhering to the mucosal tissue of the nasal passages, thus stopping further transmission. The composition of the present invention is particularly effective in preventing the virus infection because it targets the nasopharynx which has been identified as the main entry point for the virus, and forms a physical barrier like film on the surface of the nasopharynx.

### Example 4

This example is to provide formulations prepared with the composition according to the present invention. With the components described in the following tables, various formulations were prepared for administration of the composition according to the present invention.

Nasal Sprays

| Ingredients | Percent by weight |
|---|---|
| ELAH (20%) | 0.1-2%(w/v %) |
| Copper gluconate | 0.001-1.0% |
| Micro crystalline cellulose | 0.5 |
| Polysorbate80 | 0.05 |
| Phenoxy ethanol | 0.1% |
| NaOH | to adjust pH 6.5 |
| Purified water | to 100 |

Nasal Gels

| Ingredients | Percent by weight |
|---|---|
| ELAH (20%) or BAC | 0.01-5% |
| Copper gluconate | 0.001-1.0% |
| Hydroxyethylcellulose | 0.5 |
| Xylitol | 5% |
| NaOH | to adjust pH 6.5 |
| Purified water | to 100 |

Lozenges

| Ingredients | Percent by weight |
|---|---|
| ELAH (20%) or BAC | 0.01-5% |
| Copper gluconate | 0.001-1.0% |
| Xylitol | 5% |
| Flavor | 1 |
| Binder | To 100 |

Mouth Gargle

| Ingredients | Percent by weight |
|---|---|
| ELAH (20%) or BAC | 0.01-5% |
| Copper gluconate | 0.001-1.0% |
| Glycerol | 10 |
| Pluronic F108 | 0.2 |
| Flavor | 1 |
| NaOH | to adjust pH 6.5 |
| xylitol | 5% |
| Purified water | to 100 |

Surface Treatments

| Ingredients | Percent by weight |
|---|---|
| ELAH | 0.01-5% |
| Copper gluconate | 0.01-5% |
| Fragrance | 0.05 |
| HCO 40 | 0.1 |
| phenoxyethanol at | 0.1% |
| Ethanol | 10 |
| NaOH | to adjust pH 6.5 |
| Purified water | to 100 |

It will be apparent to those skilled in the art that various modifications and variations can be made in the methods and compositions of the present invention without departing from the spirit or scope of the invention. Thus, it is intended that the present invention covers the modifications and variations of this invention, provided they come within the scope of the appended claims and their equivalents.

What is claimed:

1. An antiviral microemulsion composition comprising an effective amount of an arginine ester cationic surfactant, a copper salt and a solvent.

2. The antiviral microemulsion composition of claim 1, wherein the cationic surfactant is in an amount of 2 ppm to 20,000 ppm, and the copper salt is in an amount of 1 ppm to 10,000 ppm.

3. The antiviral microemulsion composition of claim 1, wherein the solvent is selected from the group consisting of water, alcohol, propylene glycol, ethyl acetate, methyl isobutyl ketone, acetone, tetrahydrofuran, isopropyl ether, and a combination thereof.

4. The antiviral microemulsion composition of claim 1, further comprising 0.01% to 20% of a plasticizer selected from the group consisting of glycol, glycerin, xylitol, ethanol, and a combination thereof.

5. The antiviral microemulsion composition of claim 1, wherein the arginine ester cationic surfactant is ethyl lauroyl arginine hydrochloride (ELAN).

6. The antiviral microemulsion composition of claim 1, wherein the copper salt comprises a gluconate, a citrate, an acetate, an amino acid or a peptide.

7. The antiviral microemulsion composition of claim 1, wherein the cationic surfactant and the copper salt meet Equation 1 and the composition has a fractional inhibitory index (FICI) of less than 0.5:
FICI=FICA+FICB, [Equation 1]
wherein FICA=[CA]sy/[CA]al and FICB=[CS]sy/[CS]al,
wherein [CA]al is a minimum inhibitory concentration (MIC) of the cationic agent, [CS]al is a minimum inhibitory concentration (MIC) of the copper salt,
[CA]sy is a minimum inhibitory concentration (MIC) of the cationic agent where the cationic and the copper agents are used at the same time, [CS]sy is a minimum inhibitory concentration (MIC) of the copper salt where the cationic the a copper salt are used at the same time.

8. The antiviral microemulsion composition of claim 1, wherein the composition has a pH between pH 4 and pH 8.

9. The antiviral microemulsion composition of claim 1, wherein the composition is a topical formulation comprising a nasal spray, a nasal gel, an aerosol, a throat lozenge, a gargle, or an oral strip,

10. The antiviral microemulsion composition of claim 1, wherein the composition is applied to a surface in an amount of 0.01 to 100 mg/dm².

11. The antiviral microemulsion composition of claim 1, wherein the microemulsion has an average diameter of 10 to 200 nm.

12. A method of preventing or treating viral infection in a subject in need thereof, comprising applying the microemulsion composition of claim 1 to the subject.

13. The method of claim 12, wherein the composition is applied to nasal cavity comprising nostrils, nasopharynx or throat of humans and animals.

14. The method of claim 12, wherein the composition is a topical formulation comprising a nasal spray, a nasal gel, an aerosol, a throat lozenge, a gargle, an oral strip.

15. The method of claim 12, wherein the composition is applied in an amount of 0.01 to 100 mg/dm².

16. The method of claim 12, wherein the composition is pre-treated to the subject for prophylactic effect, reducing viral entry and cytopathic effect thereof.

17. The method of claim 12, wherein the composition allows a prolonged retention of the arginine ester cationic surfactant in nasal cavity.

18. The method of claim 17, wherein the arginine ester cationic surfactant is retained in nasal cavity for at least 2 hours.

19. The method of claim 17, wherein the composition forms a physical barrier on the surface of nasal cavity.

20. The method of claim 19, wherein the composition forms the physical barrier on the surface of nasopharynx.
